(19) United States
(12) Patent Application Publication
(10) Pub. No.: US 2002/0138856 A1
(43) Pub. Date: Sep. 26, 2002

(54) Compositions and methods useful for treatment of depressive disorder based on an animal model

(75) Inventors: Eva Redei, Chicago, IL (US); Fraser Aird, Oak Park, IL (US); Leah Solberg, Chicago, IL (US); Claire Will, Chicago, IL (US)

Correspondence Address: MORGAN, LEWIS & BOCKIUS LLP, 1701 MARKET STREET, PHILADELPHIA, PA, 19103-2921, US

(73) Assignee: Northwestern University

(21) Appl. No.: 10/023,335

(22) Filed: Dec. 17, 2001

Related U.S. Application Data
(63) Continuation-in-part of application No. 09/590,837, filed on Jun. 9, 2000, now abandoned.
Application No. 09/590,837 is a 371 of international application No. PCT/US99/17513, filed on Aug. 3, 1999.
(60) Provisional application No. 60/105,459, filed on Oct. 23, 1998.

(51) Int. Cl. A01K 67/027

(52) U.S. Cl. 800/3; 800/14

(57) ABSTRACT

The invention includes WKY rats which are selectively bred based on more depressed (WMI) or less depressed (WLI) behavior. The invention also includes methods of using the rats for diagnosis and treatment of behavioral disorders and further includes compositions based on the genotype differences between WMI and WLI rats.

### CROSS-REFERENCE TO RELATED APPLICATIONS

This application is a continuation-in-part of U.S. application Ser. No. 09/590,837, filed Jun. 9, 2000, which claims priority to International Application No. PCT/US99/17513, filed Aug. 3, 1999, and to U.S. application Ser. No. 60/105,459, filed Oct. 23, 1998, all of which are hereby incorporated by reference.

### BACKGROUND OF THE INVENTION

Depression is a major illness in humans which is believed to have a genetic basis, at least in some instances. However, there is a paucity of information regarding the genes and protein products which govern depressive events in humans.

Stressful life events are known to precipitate depressive episodes in some people but not in others. The vulnerability to the depression-inducing effects of stress may originate from genetic predisposition to depression and/or from specific hypersensitivity to the effects of stress in these individuals.

To date, there are very few animal models available for the study of depression and there are no available animal models which are well characterized genetic models for endogenous depression. Okamoto and Aoki (1963, Jpn. Circ. J. 27:282-293) isolated a strain of Wistar rats exhibiting spontaneously developed hypertension, the spontaneously hypertensive rat (SHR). The normotensive inbred progenitor strain of the SHR, the Wistar Kyoto (WKY) rat differs from the SHR with respect to resting blood pressure, and further, the WKY rat displays larger endocrine and behavioral responses to stress compared with these responses in SHRs, the parent Wistar rats and several other rat strains (Pare and Redei, 1995, In: Neuroendocrinology of Gastrointestinal Ulceration (S. Szabo and Y. Tache eds), Plenum Press, New York. pp 201-208). A large body of literature obtained in different laboratories supports these findings. WKY rats exhibit increased depressive and anxious behavior in a number of tests compared with a number of inbred and outbred rat strains (Pare and Redei, 1995, In: Neuroendocrinology of Gastrointestinal Ulceration (S. Szabo and Y. Tache eds), Plenum Press, New York. pp 201-208; Lahmane and Armario, 1996, Psychopharmacology 123:191-198; Lahmane et al., 1997, Eur. J. Pharmacol. 337:115-123). The fact that these animals exhibit both depressive and anxious behavior mirrors human psychopathology where there is also observed a substantial co-morbidity of depression and anxiety disorders (Pini et al., 1997, J. Affective Disorders 42:145-153). Acute administration of selective serotonin reuptake inhibitors (SSRI) or low dose of other antidepressants had no effect on depressive behavior in WKY rats, while it did in other rat strains (Lahmane et al., 1997, Eur. J. Pharmacol. 337:115-123). Chronic and subacute (treatment administered three times per day between first and third swim) treatment with imipramine or with desipramine decreased the depressive behavior of WKY rats as measured by decreased immobility and increased struggling in the forced swim test (Pare and Redei, 1993, J. Physiol. 87:229-238; Lahmane and Armario, 1996, Psychopharmacology 123:191-198). The reduced sensitivity of WKY rats to antidepressant treatment prompted Lahmane et al., (1996, 1997) to propose that they are a model of treatment-resistant depression. However, others found no difference in the sensitivity of WKY rats to desipramine treatment (Lopez-Rubalcava and Lucki, 2000, Neuropsychopharmacology 22:191-199). Dysregulation of the hypothalamic-pituitary-adrenoncortical (HPA) axis is the most heavily examined area in research involving clinical neuroendocrinology of affective disorders (Holsboer and Barden, 1996, Endocr. Rev. 17:187-204). The most common findings suggest a general hyperactivity of the HPA axis that is particularly prevalent in melancholic depression and also in anxiety disorders. This hyperactive HPA function is characterized by hypercortisolemia. Patients having depressive disorder, particularly of the melancholic type, often exhibit elevated plasma cortisol levels, which are usually measured at the circadian peak, i.e., in the morning in humans. WKY rats also exhibit hyperactivity of the HPA axis, primarily at the level of the anterior pituitary. Increased steady-state levels of proopiomelanocortin (POMC) mRNA (Redei et al., 1994, Am. J. Physiol. 266:R353-R360) and an exaggerated adrenocorticotropin (ACTH) response to acute stress in WKY rats compared with Wistar and Fisher 344 rats has been reported (Pare and Redei, 1993, J. Physiol. 87:229-238; Redei et al., 1994, Am. J. Physiol. 266:R353-R360).

Notwithstanding the above, the WKY rat exhibits a varied profile with respect to behavior responsiveness to antidepressant treatment and depressive-related physiology and therefore has some limitations with respect to its use as a genetic animal model. There is a need in the art for a more reliable animal model for the study of depression. The present invention satisfies this need.

### SUMMARY OF THE INVENTION

The invention includes a WMI rat, wherein the rat exhibits a forced swim test immobility score of greater than about 9. Preferably, the WMI rat exhibits a forced swim test score of greater than about 11.

The invention also includes a WLI rat, wherein the rat exhibits a forced swim test immobility score of lower than about 8. Preferably, the WLI rat exhibits a forced swim test immobility score of lower than about 6.

Also included in the invention is a method of identifying a compound capable of treating a behavioral disorder in a mammal. The method comprises administering a test compound to a WMI rat, administering a placebo to an otherwise identical WMI rat, and assessing the behavior of each of the rats, wherein a change in behavior of the rat administered the test compound, compared with the behavior of the rat administered the placebo, is an indication that the test compound is capable of treating a behavioral disorder in a mammal.

In one aspect, the mammal is resistant to classical antidepressant drugs, including, but not limited to tricyclic and serotonergic antidepressants.

In one aspect, the compound is a fast-acting compound.

In one aspect, the treatment is alleviation of behavioral disorder.

In another aspect, the behavior is assessed in a test selected from, but not restricted to the group consisting of a forced swim test, an elevated plus maze test, a light/dark box test, an open field behavior test and a locomotor behavior test.

In another aspect, the mammal is a human and when the mammal is a human, the behavioral disorder is selected from the group consisting of major depression, bipolar depression, generalized anxiety disorder, obsessive compulsive disorder, post-traumatic stress disorder, dysthymia, cyclothymia and premenstrual syndrome.

Also included in the invention is a method of identifying a compound capable of treating a behavioral disorder in a mammal. The method comprises administering a test compound to a WLI rat, administering a placebo to an otherwise identical WLI rat, and assessing the behavior of each of the rats, wherein a change in behavior of the rat administered the test compound, compared with the behavior of the rat administered the placebo, is an indication that the test compound is capable of treating a behavioral disorder in a mammal.

In one aspect, the mammal is resistant to classical antidepressant drugs, including, but not limited to tricyclic and serotonergic antidepressants.

In one aspect, the compound is a fast-acting compound.

In one aspect, the treatment is alleviation of behavioral disorder.

In another aspect, the behavior is assessed in a test selected from, but not restricted to the group consisting of a forced swim test, an elevated plus maze test, a light/dark box test, an open field behavior test and a locomotor behavior test.

In another aspect, the mammal is a human and when the mammal is a human, the behavioral disorder is selected from the group consisting of major depression, bipolar depression, generalized anxiety disorder, obsessive compulsive disorder, post-traumatic stress disorder, dysthymia, cyclothymia and premenstrual syndrome.

Also included in the invention is a method of isolating a nucleic acid associated with a behavioral disorder in a mammal. The method comprises comparing mRNA obtained from a tissue in a WMI rat with mRNA obtained from the same tissue in a WLI rat and isolating nucleic acid corresponding to mRNA expressed in the WMI rat, which mRNA is either not expressed in the WLI rat or is expressed at a substantially lower level in the WLI rat, thereby isolating a nucleic acid associated with a behavioral disorder in mammal.

In one aspect, the mammal is resistant to classical antidepressant drugs, including, but not limited to tricyclic and serotonergic antidepressants.

In one aspect, the comparison of mRNAs is conducted by differential screening of libraries which are generated using the mRNAs.

In another aspect, the comparison is conducted using reverse transcriptase PCR differential display. In a preferred embodiment, the comparison of mRNAs is conducted by the method of suppressive, subtractive PCR.

In yet another preferred embodiment, the comparison of mRNAs is conducted using a DNA chip analysis microarray.

In a preferred embodiment, the tissue is selected from the group consisting of whole brain, amygdala, prefrontal cortex, hippocampus, and other limbic brain region. More preferably, the tissue is amygdala, prefrontal cortex, or hippocampus.

In one aspect, the nucleic acid which is isolated using the method above has a nucleotide sequence selected from the group consisting of SEQ ID NO: 1 and SEQ ID NO: 3.

Also included in the invention is a method of isolating a nucleic acid associated with a behavioral disorder in a mammal. The method comprises comparing mRNA obtained from a tissue in a WMI rat with mRNA obtained from the same tissue in a WLI rat and isolating nucleic acid corresponding to mRNA which is either not expressed in the WMI rat or which is expressed in the WMI rat at a substantially lower level than expression of the nucleic acid in the WLI rat, thereby isolating a nucleic acid associated with a behavioral disorder in a mammal.

In one aspect, the mammal is resistant to classical antidepressant drugs, including, but not limited to tricyclic and serotonergic antidepressants.

In one aspect, the comparison of the mRNAs is conducted by differential screening of libraries which are generated using the mRNAs.

In another aspect, the comparison is conducted using reverse transcriptase PCR differential display. In a preferred embodiment, the comparison of mRNAs is conducted using suppressive, subtractive PCR.

In a preferred embodiment, the tissue is selected from the group consisting of whole brain, amygdala, prefrontal cortex, hippocampus, and limbic brain region. More preferably, the tissue is amygdala, prefrontal cortex, or hippocampus.

Also included in the invention is a nucleic acid associated with a behavioral disorder in a mammal, wherein the nucleic acid is isolated by the method of comparing mRNA obtained from a tissue in a WMI rat with mRNA obtained from the same tissue in a WLI rat and isolating nucleic acid corresponding to mRNA expressed in the WMI rat, which mRNA is either not expressed in the WLI rat or is expressed at a substantially lower level in the WLI rat, thereby isolating the nucleic acid.

In one aspect, the mammal is resistant to classical antidepressant drugs, including, but not limited to tricyclic and serotonergic antidepressants.

In addition, the invention includes an antisense nucleic acid which corresponds to a nucleic acid associated with a behavioral disorder in a mammal, wherein the nucleic acid is isolated by the method of comparing mRNA obtained from a tissue in a WMI rat with mRNA obtained from the same tissue in a WLI rat and isolating nucleic acid corresponding to mRNA expressed in the WMI rat, which mRNA is either not expressed in the WLI rat or is expressed at a substantially lower level in the WLI rat, thereby isolating the nucleic acid.

In one aspect, the mammal is resistant to classical antidepressant drugs, including, but not limited to tricyclic and serotonergic antidepressants.

The invention further includes a nucleic acid associated with a behavioral disorder in a mammal, wherein the nucleic acid is isolated by the method of comparing mRNA obtained from a tissue in a WMI rat with mRNA obtained from the same tissue in a WLI rat and isolating nucleic acid corresponding to mRNA which is either not expressed in the WMI rat or which is expressed in the WMI rat at a substantially lower level than expression of the nucleic acid in the WLI rat, thereby isolating the nucleic acid.

In one aspect, the mammal is resistant to classical antidepressant drugs, including, but not limited to tricyclic and serotonergic antidepressants.

In addition, the invention includes an antisense nucleic acid which corresponds to nucleic acid associated with a behavioral disorder in a mammal, wherein the nucleic acid is isolated by the method of comparing mRNA obtained from a tissue in a WMI rat with mRNA obtained from the same tissue in a WLI rat and isolating nucleic acid corresponding to mRNA which is either not expressed in the WMI rat or which is expressed in the WMI rat at a substantially lower level than expression of the nucleic acid in the WLI rat, thereby isolating the nucleic acid.

In one aspect, the mammal is resistant to classical antidepressant drugs, including, but not limited to tricyclic and serotonergic antidepressants.

Also included in the invention is a method of isolating a polypeptide associated with a behavioral disorder in a mammal. The method comprises comparing mRNA obtained from a tissue in a WMI rat with mRNA obtained from the same tissue in a WLI rat and isolating nucleic acid corresponding to mRNA expressed in the WMI rat, which mRNA is either not expressed in the WLI rat or is expressed at a substantially lower level in the WLI rat, thereby isolating a nucleic acid associated with a behavioral disorder in the mammal and obtaining the polypeptide encoded thereby.

In one aspect, the mammal is resistant to classical antidepressant drugs, including, but not limited to tricyclic and serotonergic antidepressants.

In one aspect, the comparison of mRNAs is conducted by differential screening of libraries which are generated using the mRNAs.

In another aspect, the comparison is conducted using reverse transcriptase PCR differential display. In a preferred embodiment, the comparison of mRNAs is conducted by the method of suppressive, subtractive PCR.

In a preferred embodiment, the tissue is selected from the group consisting of whole brain, amygdala, prefrontal cortex, hippocampus, and limbic brain region.

The invention also includes a polypeptide associated with a behavioral disorder in a mammal isolated by the method of comparing mRNA obtained from a tissue in a WMI rat with mRNA obtained from the same tissue in a WLI rat and isolating nucleic acid corresponding to mRNA expressed in the WMI rat, which mRNA is either not expressed in the WLI rat or is expressed at a substantially lower level in the WLI rat, thereby isolating a nucleic acid associated with a behavioral disorder in the mammal and obtaining the polypeptide encoded thereby.

In one aspect, the mammal is resistant to classical antidepressant drugs, including, but not limited to tricyclic and serotonergic antidepressants.

The invention further includes an antibody which specifically binds to a polypeptide isolated by the method of comparing mRNA obtained from a tissue in a WMI rat with mRNA obtained from the same tissue in a WLI rat and isolating nucleic acid corresponding to mRNA expressed in the WMI rat, which mRNA is either not expressed in the WLI rat or is expressed at a substantially lower level in the WLI rat, thereby isolating a nucleic acid associated with a behavioral disorder in the mammal and obtaining the polypeptide encoded thereby.

In one aspect, the mammal is resistant to classical antidepressant drugs, including, but not limited to tricyclic and serotonergic antidepressants.

The invention additionally includes a method of isolating a polypeptide associated with a behavioral disorder in a mammal. The method comprises comparing mRNA obtained from a tissue in a WMI rat with mRNA obtained from the same tissue in a WLI rat and isolating nucleic acid corresponding to mRNA which is either not expressed in the WMI rat or which is expressed in the WMI rat at a substantially lower level than expression of the nucleic acid in the WLI rat, thereby isolating a nucleic acid associated with a behavioral disorder in the mammal and obtaining the polypeptide encoded thereby.

In one aspect, the mammal is resistant to classical antidepressant drugs, including, but not limited to tricyclic and serotonergic antidepressants.

In one aspect, the comparison of mRNAs is conducted by differential screening of libraries which are generated using the mRNAs.

In another aspect, the comparison is conducted using reverse transcriptase PCR differential display. In a preferred embodiment, the comparison of mRNAs is conducted by the method of suppressive, subtractive PCR.

In a preferred embodiment, the tissue is selected from the group consisting of whole brain, amygdala, prefrontal cortex, hippocampus, and limbic brain region.

Also included in the invention is a polypeptide associated with a behavioral disorder in a mammal, wherein the polypeptide is isolated by the method of comparing mRNA obtained from a tissue in a WMI rat with mRNA obtained from the same tissue in a WLI rat and isolating nucleic acid corresponding to mRNA which is either not expressed in the WMI rat or which is expressed in the WMI rat at a substantially lower level than expression of the nucleic acid in the WLI rat, thereby isolating a nucleic acid associated with a behavioral disorder in the mammal and obtaining the polypeptide encoded thereby.

In one aspect, the mammal is resistant to classical antidepressant drugs, including, but not limited to tricyclic and serotonergic antidepressants.

Further included in the invention is an antibody which specifically binds to a polypeptide isolated by the method of comparing mRNA obtained from a tissue in a WMI rat with mRNA obtained from the same tissue in a WLI rat and isolating nucleic acid corresponding to mRNA which is either not expressed in the WMI rat or which is expressed in the WMI rat at a substantially lower level than expression of the nucleic acid in the WLI rat, thereby isolating a nucleic acid associated with a behavioral disorder in the mammal and obtaining the polypeptide encoded thereby.

In one aspect, the mammal is resistant to classical antidepressant drugs, including, but not limited to tricyclic and serotonergic antidepressants.

The invention additionally includes an isolated cell obtained from a WMI rat and a cell obtained from a WLI rat.

### BRIEF DESCRIPTION OF THE DRAWINGS

FIG. 1 is a graph depicting selective breeding of 10 week-old most immobile (WMI) and least immobile (WLI) rat substrains. Black squares indicate WMI male rats, unshaded squares indicate WMI female rats, shaded diamonds indicate WLI male rats, and unshaded diamonds indicate WLI female rats.

FIG. 2 is a graph depicting the number of lines crossed (ambulation) in the open field test of F1 generation WMI and WLI rats.

FIG. 3 is a graph depicting the plasma corticosterone profile of FI generation WMI and WLI rats.

FIG. 4 is a graph depicting the plasma TSH profile of F1 generation WMI and WLI male rats.

FIG. 5 is a series of gels depicting differential display RT-PCR analysis which compares the amygdala RNA obtained from the parental generation of breeding mothers in WMI and WLI rats.

FIG. 6 is a graph depicting the immobility in the forced swim test of 14 week-old animals before and after administration of either desipramine, fluoxetine, or water in WMI and WLI animals. Shaded squares and circles indicate immobility after treatment, unshaded squares and circles indicate immobility before treatment, squares indicate WMI animals, and circles indicate WLI animals.

FIG. 7 is a scatter plot depicting variability in the immobility measure of the forced swim test in each group of F344 (inbred), Wistar (outbred), and WKY (inbred) rats. Shaded diamonds with standard deviation bars indicate the mean variability for each group. WKY rats demonstrated more immobility and greater variability. FIG. 8, comprising FIGS. 8A and 8B, lists the nucleotide sequence of rat mRNA for mss4 protein (GenBank Accession No. X70496; SEQ ID NO: 1).

FIG. 9 lists the mRNA nucleotide sequence of human mss4 protein (GenBank Accession No. S78873; SEQ ID NO: 2).

FIG. 10 lists the nucleotide sequence of rat mitochondrial ATP synthase subunit 6 (GenBank Accession No. AF115770; SEQ ID NO: 3).

FIG. 11 lists the nucleotide sequence of human mitochondrial ATP synthase subunit 6 (GenBank Accession No. AF346963; SEQ ID NO: 4).

### DETAILED DESCRIPTION

The invention is based on the discovery of an animal model for the study of depression and other behavioral disorders. According to the invention, founder rats have been generated which are capable of breeding to generate progeny rats, which founders and progeny exhibit highly depressive behavior. The generation of highly depressed and non-depressed rats from depressed rats was surprising because it was expected that Wistar Kyoto rats are a homogenous inbred strain that would behave uniformly.

There is a long felt need in the art for compounds which alleviate depression or other behavioral disorders in an animal in a “fast-acting” manner. Currently available therapeutic compounds used to treat depression require several days to several weeks before the symptoms are alleviated. The animal model of the present invention is therefore particularly useful for the identification and characterization of compounds which provide immediate relief from symptoms of depression and other behavioral disorders.

By the term “fast-acting” as used herein is meant a compound which alleviates the symptoms of a behavioral disorder in the animal in a shorter period of time than currently commercially available compounds. Since the majority of currently available compounds used, particularly for treatment of depression, take several days to weeks to have maximal effect, a “fast-acting” compound is considered to be one which exerts its effect on the disease or disorder in about one to two days or less.

In the experiments performed in the present invention, Wistar Kyoto (WKY) rats were segregated into two groups, more depressed and less depressed rats, and selectively bred to acquire WMI (WKY, more immobile) and WLI (WKY, less immobile) rats. This was accomplished using the methods for selective breeding disclosed in U.S. Pat. No. 5,602,302. Briefly, the methods disclosed therein were used to generate guinea pigs that were hypersensitive or hyposensitive to certain inhalant chemicals. Parental guinea pig strains were separated based on their responsiveness to certain inhalants. These characteristics were used to breed two subsequent populations of guinea pig with distinct behaviors. Similarly, the parental strain were segregated into two groups using the forced swim test, discussed in more detail below, to determine depressive characteristics. The more depressed rats were bred with each other, and the less depressed rats were also bred with each other. At about 60 days of age, i.e., as young adults, the rats were tested for depressive behavior as described in detail herein, and those progeny rats exhibiting more depressive behavior were selected for further breeding to generate a colony of WKY-More Immobile (WMI) rats. Similarly, those progeny rats exhibiting less depressive behavior were also selected for further breeding to generate a colony of WKY-Less Immobile (WLI) rats. At least F1 generation, and preferably F2 generation colonies of WMI and WLI rats were thus obtained which are the subject of the present invention.

The invention thus includes two types of inbred rats which are referred to herein as WMI and WLI rats. WMI rats are those which exhibit depressive behavior and which have a forced swim test (FST) immobility score which is higher than about 9. Preferably, a WMI rat has a FST immobility score of higher than about 11. Additionally, WMI rats are responsive to desipramine when it is administered three times within 24 hours. WLI rats are those which do not exhibit depressive behavior and which have an FST immobility score which is lower than about 8, and preferably, WLI rats have a FST immobility score which is lower than about 6. Contrary to the WMI rat, WLI rats are do not respond to desipramine treatment. WMI and WLI rats are the progeny of WKY rats having an FST immobility score of more than about 9 in the case of WMI rats, and those which are the progeny of WKY rats having an FST immobility score of lower than about 8, in the case of WLI rats. The rats of the invention must be at least the F1 progeny of commercially available WKY rats selected for breeding on the basis of depressive behavior, and preferably, the rats are F2 or higher progeny.

WMI and WLI rats do not exist in nature in their pure form as each has been bred such that individuals within each substrain exhibit identical behavioral phenotypes which are distinct from those exhibited by individuals in the other strain. Since the phenotypes are genetically transmissible, the two substrains must be genetically distinct from each other. Thus, these geneticaly segregated substrains are not subject to wide variation in behavior and responsiveness. This is contrary to the parental WKY generation, which is subject to wide variations in behavior and responsiveness tests (forced swim test, effects of anti-depressant drugs, hormone levels) as compared with inbred Fisher 344 rats and outbred Wistar rats (FIG. 8). The WLI and WMI breeds, therefore, are superior for predicting outcomes of behavior and responsiveness results based on the genetic identity of the strain.

Assessment of depressive behavior in inbred F1 or higher generation WMI or WLI rats is conducted by measuring their score in the FST. Other behavioral parameters which may be used are the elevated plus maze test, the light/dark box test, the open field behavior test and the locomotor behavior test. In addition, the circadian profile of plasma ACTH and corticosterone as well as thyroid stimulating hormone (TSH) are measured. This list of tests should in no way be construed as being all inclusive; rather, any suitable test for behavior which is routinely practiced may be included as an assessment of the behavior of the animals of the invention.

The main test of behavior in the rats of the invention is the FST. The general parameters governing this test are as follows, it being understood that variations of the test are permissible within acceptable bounds. The test utilizes a glass water tank 30 cm in diameter and 45 cm tall wherein the water level is 15 cm from the top and the water temperature is 25° C. The procedure for the test is adapted from Porsolt et al. and Detke et al., as described in Detke et al., 1995, Psychopharm. 121:66-72). Rats are individually placed in the water tank for 15 minutes and are then removed from the tank and returned to their cages. Twenty four hours later, the rats are placed once more in the tank for 5 minutes. This 5 minute test session is recorded on videotape. The performance of the rat on the tape is analyzed and subsequently scored using the scoring system developed by Detke et al. (1995, Psychopharm. 121:66-72) by a trained observer, who is unaware as to the various treatment conditions of the rats. In other words, the scores are generated in a “blind” study fashion. The following behaviors are recorded: immobility, swimming, and climbing. The rats are scored at 5 second intervals, thus, there are 12 intervals per minute sampled. Including the first interval at time 0, there is a maximum total of 61 measurements taken for each animal. By way of example, if a rat was scored as immobile at 13 of the 61 intervals, the FST score would be 13. The rats are then towel dried, placed into a heated cage for at least 15 minutes and are subsequently returned to their home cages. As noted above, rats having FST immobility scores of greater than about 9 are considered to be the WMI rats of the invention and rats having FST immobility scores of lower than about 8 are considered to be the WLI rats of the invention, provided these rats are at least the F1 progeny, and preferably F2 progeny or higher, of commercially available WKY rats.

Other suitable tests of behavior are as follows. The results of each test are interpreted according to the standards in the art.

The elevated plus maze test is conducted as follows. Basal Plus Maze performance assesses anxiety-related risk-taking behavior in the rat. Animals are placed onto the center platform of the elevated plus maze facing a closed arm. The 5 minute test session is videotaped and subsequently scored by a trained observer who is blind to the treatment conditions. The following parameters are measured: The latency to enter the first arm; the number of open and closed arm entries and the total time spent in the open and closed arms.

The locomotor activity test is performed as follows. The locomotor chambers are clear Plexiglass boxes placed inside an activity monitor containing beams of light from which activity is sensed. The activity monitor is equipped with optical sensors. Sequential beam disruption is required to register locomotor activity. Activity (5 minutes) is recorded by a computer using the data collection software.

The open field behavior test is conducted as follows. Animals are placed in the center of a novel 15 inch diameter open-field and behavior is videotaped for 10 minutes. The circular open-field is enclosed with an 18 inch high wall. The floor is divided into four quadrants for assessment of movement within the field. Illumination is approximately 160 lux. The measures scored include activity, rearing, and grooming quantified for the entire examination period.

The light/dark box test is conducted as follows. Behavior in the light/dark box assesses anxiety-related behavior in the rat. The light/dark box is divided into an 18 inch long×15 inch wide×15 inch high compartment open at the top having three walls. The floor of the box is made of clear Plexiglass. The fourth wall serves as the divider between the two compartments and contains a 3 inch wide×4 inch high opening at floor level. This allows the animal to enter into a 12 inch long×15 inch wide dark compartment which is fully enclosed. The four walls, the ceiling and the floor of the dark compartment are made of black Plexiglass. A door at the back of the box serves to allow access for cleaning and removal of the animals after testing. The test apparatus is placed on a 30 inch deep bench top. At the beginning of test, each animal is placed in the center of the light compartment. Behavior is subsequently videotaped for 15 minutes. Behaviors are scored by an observer who is blind to the treatment conditions. The parameters which are measured are initial latency to enter the dark compartment, number of compartment entries, and the total time spent in each compartment. The average lighting in the testing room at the level of the maze is 200 lux.

As the data presented in the experimental Example disclosed herein indicates, the WMI rat exhibits a genetic vulnerability to stress. Thus, the WMI rat of the invention is also an animal model for this type of behavioral disorder.

The WMI and WLI rats of the invention are useful for screening compounds effective for modulation, preferably, alleviation of depression and other behavioral and depressive disorders. Such screening is accomplished as follows. WMI and WLI rats are administered a test compound or a placebo at a dosage, a route of administration and for a period of time which is determined by the artisan skilled in such procedures, following standard protocols such as those described, for example, in Remington's Pharmaceutical Sciences, Mack Publishing Co., Easton, Pa., 1985. The behavior of the rats is assessed using any number of the criteria disclosed herein, including, most particularly, the FST. The effect of the test compound on, for example, the FST immobility score of any given group of rats is assessed to determine whether the test compound modulates the behavior of the rats. An increase in the FST immobility score in rats (both WMI and WLI) following administration of the test compound compared with the FST immobility score in otherwise identical rats to whom the placebo is administered, is an indication that the test compound promotes depression or other behavioral disorders. In contrast, a decrease in the FST immobility score in either of the WMI or the WLI rats following administration of the test compound, compared with the FST immobility score in otherwise identical rats to whom the placebo is administered, is an indication that the test compound alleviates depression or other behavioral disorders.

Compounds identified by the method of the invention which affect behavior in the rats are preferably those which have a rapid positive effect on depression and other behaviors in the rat, in that the compounds rapidly alleviate the symptoms of these diseases or disorders.

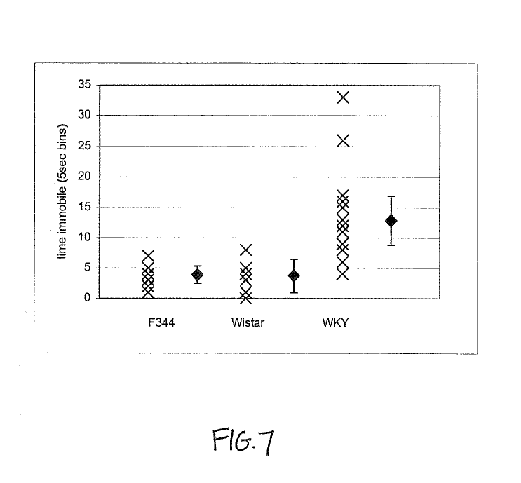

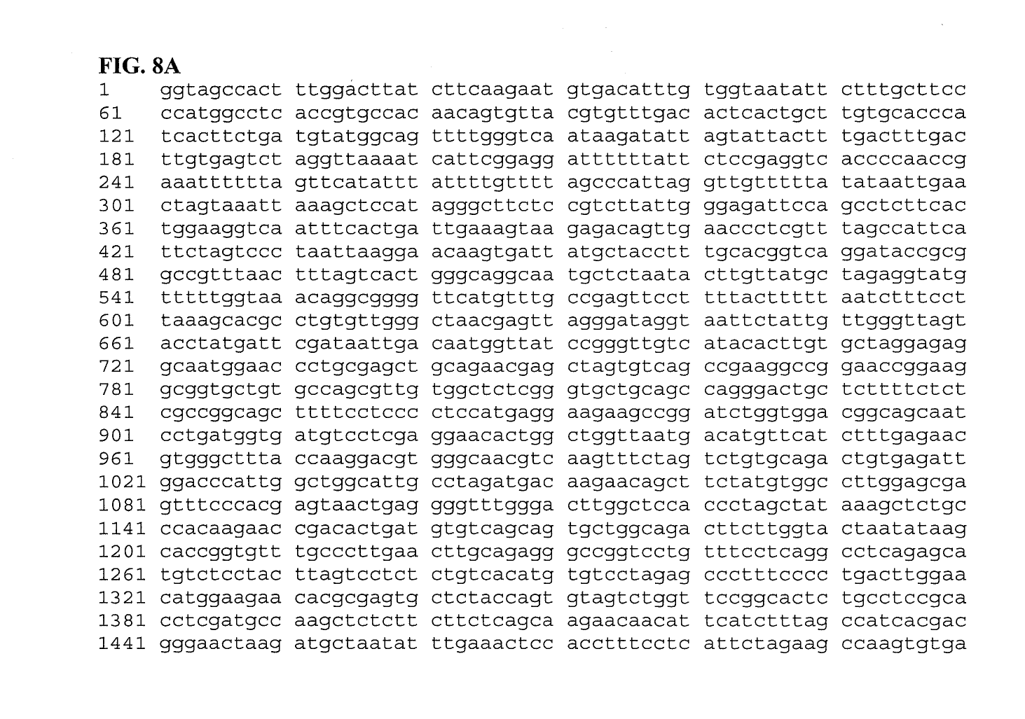

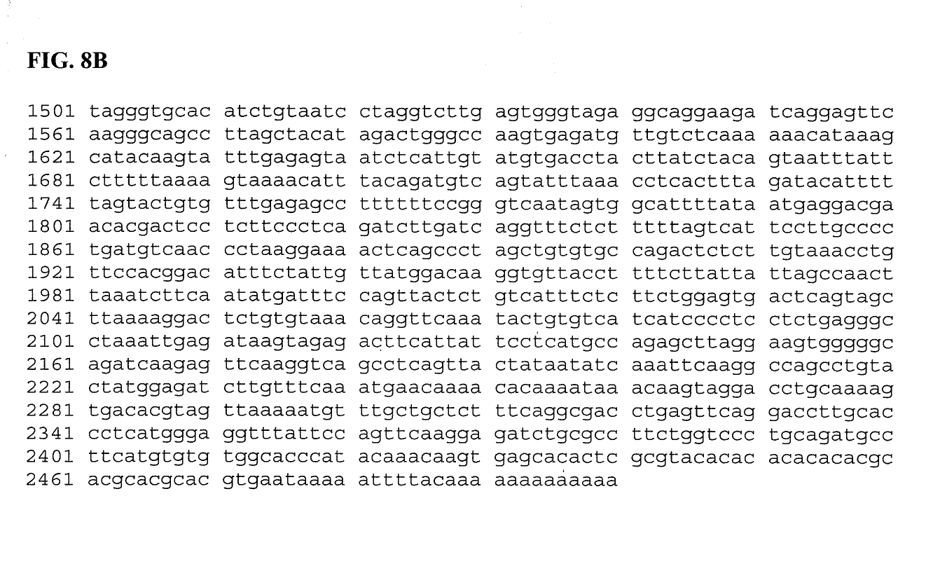

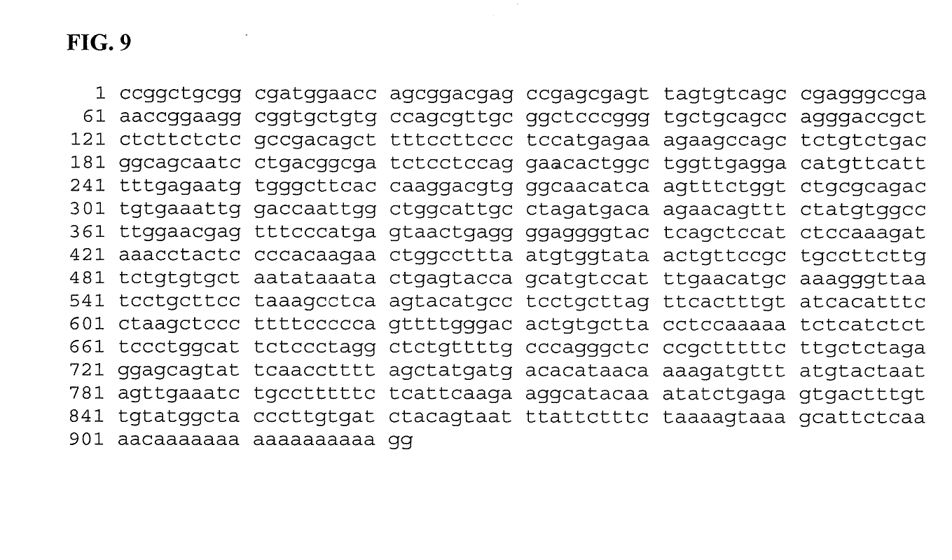

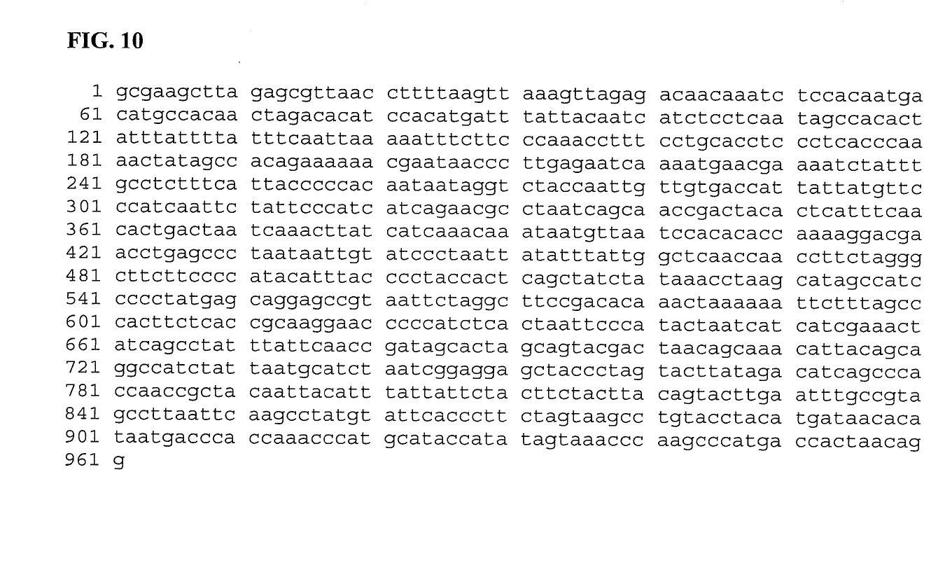

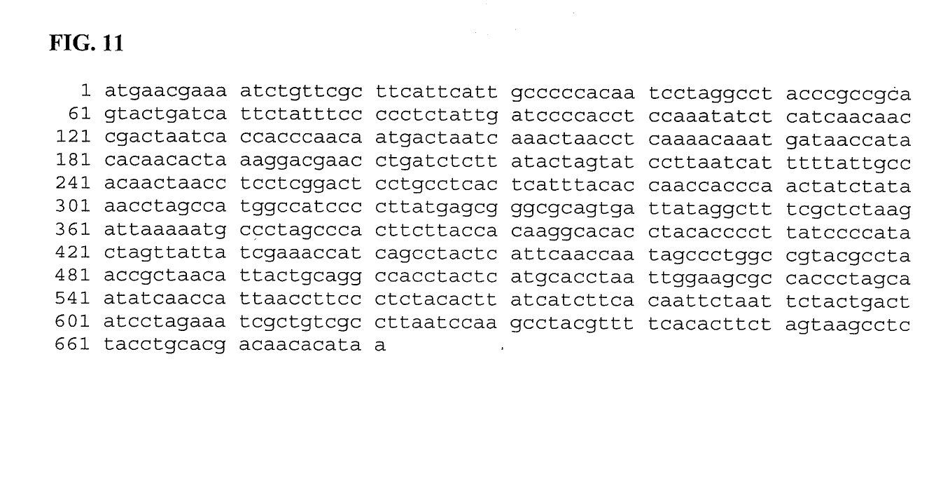

FIG. 6 demonstrates the effectiveness of two compounds on alleviating depression as scored by the forced swim test. Desipramine, which affects the norepinephrine uptake pathway, or fluoxetine which affects the serotonin uptake pathway, two compounds presently used for treating depression in humans, were administered to WMI and WLI rats. Measurements of immobility were taken before and after administration of the compounds. Desipramine caused the immobility time in WMI rats to drop from about nineteen 5-second intervals to about five 5-second intervals, and in WLI rats from about nine 5-second intervals to about six 5-second intervals (not significant). Thus, desipramine effectively alleviated depression in WMI but not in WLI rats, suggesting that WLI rats are resistant to treatment with desipramine. Fluoxetine had virtually no effect on alleviating depression in either strain. Without wishing to be bound by any particular theory, the lack of effect of fluoxetine may be explained by the genetic traits of the rats being segregated by the selective breeding process. Specifically, those genes involved in norepinephrine uptake and not serotonin uptake segregate with the other behavioral genes.

While one of the goals of the present invention is to identify compounds which are capable of alleviating or treating depression and other behavioral disorders, and the present screening method serves this purpose, it is also important to note that the rats of the invention are useful for testing pharmaceutical compounds for their ability to promote depression or other behavioral disorders. There is need in the art to identify this characteristic, if present, in pharmaceutical compounds which are used to treat disorders which may be unrelated to depression and behavioral disorders. Identification of the ability of a compound to promote depression or other behavioral disorders provides crucial information about that compound and the suitability of its use in treating the disease for which it was designed.The rats of the invention are also useful for the identification of genes and proteins involved in the promotion or alleviation of depression and other behavioral disorders in mammals, particularly in humans. Animal models have proven to be suitable models for human diseases (Wajnrajch, et al., 1996, Nature Genetics 12:88-90; Godfrey, et al., 1993, Nature Genetics, 4:227-32; Sipione and Cattaneo, 2001, Molecular Neurobiology, 23:21-51; Siddique et al., 1996, Trends in Genetics, 10:S7-S12; Hock and Lamb, 1996, Neurology, 46:S27-S34), thus the rats of the present invention are useful models for human depression. For example, it is now possible, given the present invention, to perform differential screening to identify genes and proteins which affect depression and other behavioral disorders in mammals.

To identify such genes and nucleic acids, separate nucleic acid libraries may be generated using mRNA obtained from WMI rats and mRNA obtained from WLI rats, named WMI and WLI libraries, respectively. Differential screening of nucleic acids is a technique which is common in the art of molecular biology and serves, via hybridization technology, to identify nucleic acids which are expressed in a cell versus the absence of expression of the same nucleic acid in another cell. Differential screening is described in the art, for example in Sambrook et al. (1989, Molecular Cloning: A Laboratory Manual, Cold Spring Harbor Laboratory, New York), in Ausubel et al. (1997, Current Protocols in Molecular Biology, John Wiley & Sons, New York) and in Gerhardt et al. eds. (1994, Methods for General and Molecular Bacteriology, American Society for Microbiology, Washington, D.C.). Nucleic acids which are expressed in a WMI rat, compared with the absence of expression of the same nucleic acid in the WLI rat, represent nucleic acids which potentially play a role in depression and other behavioral disorders. Similarly, nucleic acids which are not expressed in WMI rats, but which are expressed in WLI rats, also represent nucleic acids which potentially play a role in depression and other behavioral disorders. Also, nucleic acids which have a different level of expression in one type of rat compared with the expression of the nucleic acid in the other type of rat, represent nucleic acids which potentially play a role in depression and other behavioral disorders.

Expression WMI and WLI libraries may be generated using ordinary molecular biology technology, such as that described for example, in Sambrook et al. (1989, Molecular Cloning: A Laboratory Manual, Cold Spring Harbor Laboratory, New York), in Ausubel et al. (1997, Current Protocols in Molecular Biology, John Wiley & Sons, New York) and in Gerhardt et al. eds. (1994, Methods for General and Molecular Bacteriology, American Society for Microbiology, Washington, D.C.). Thus, the protein products of nucleic acids identified in the differential screening assay described herein may be deduced from the nucleic acid sequence of the nucleic acid, and may be obtained by expressing the nucleic acid in an expression system.

The invention thus includes a method of identifying a nucleic acid associated with depression or other behavioral disorders in a mammal. The method comprises obtaining total mRNA from a tissue in a WMI rat and an identical tissue in a WLI rat, generating an WMI and an WLI library therefrom, and performing differential screening on the library, wherein, nucleic acid expressed in a WMI rat which is not expressed in a WLI rat is nucleic acid which is associated with depression or other behavioral disorders in the mammal.

Similarly, the invention also includes a method of identifying a nucleic acid associated with depression or other behavioral disorders in a mammal. The method comprises obtaining total mRNA from a tissue in a WMI rat and an identical tissue in a WLI rat, generating an WMI and an WLI library therefrom, and performing differential screening on the library, wherein, nucleic acid which is not expressed in a WMI rat, but which is expressed in a WLI rat is nucleic acid which is associated with depression or other behavioral disorders in the mammal.

In addition, the invention includes a method of identifying a nucleic acid associated with depression or other behavioral disorders in a mammal. The method comprises obtaining total mRNA from a tissue in a WMI rat and an identical tissue in a WLI rat, generating an WMI and an WLI library therefrom, and performing differential screening on the library, wherein, nucleic acid which is expressed at a different level in one type of rat, compared with the level of expression in the other type of rat, is nucleic acid which is associated with depression or other behavioral disorders in the mammal.

The invention also includes a method of identifying a nucleic acid associated with depression or other behavioral disorders in a mammal using subtractive, suppressive PCR. The method comprises isolating mRNA from a tissue of a WMI rat and an identical tissue of a WLI rat. The mRNA is then transcribed to cDNA, and used as both tester and driver during subtractive PCR. PCR generates clones which are analyzed against probes prepared from identical tissue of both WLI and WMI rats. Clones that show greater expression in one strain versus the other strain are selected and sequenced, thereby identifying a nucleic acid which is associated with depression or other behavioral disorders in the mammal.

Similarly, the invention includes a method of identifying a protein associated with depression or other behavioral disorders in a mammal. The method comprises obtaining total mRNA from a tissue in a WMI and an identical tissue in a WLI rat, generating an WMI and an WLI library therefrom, and performing differential screening on the library, to identify a nucleic acid which is expressed in a WMI rat that is not expressed in a WLI rat and identifying the protein product encoded thereby, thereby identifying a protein which is associated with depression or other behavioral disorders in the mammal.

The invention also includes another method of identifying a protein associated with depression or other behavioral disorders in a mammal. The method comprises obtaining total mRNA from a tissue in a WMI and an identical tissue in a WLI rat, generating an WMI and an WLI library therefrom, and performing differential screening on the library, to identify a nucleic acid which is not expressed in a WMI rat that is expressed in a WLI rat and identifying the protein product encoded thereby, thus identifying a protein which is associated with depression or other behavioral disorders in the mammal.

The invention further includes yet another method of identifying a protein associated with depression or other behavioral disorders in a mammal. The method comprises obtaining total mRNA from a tissue in a WMI and an identical tissue in a WLI rat, generating an WMI and an WLI library therefrom, and performing differential screening on the library, to identify a nucleic acid which is expressed at a different level in one type of rat, compared with the level of expression of the nucleic acid in the other type of rat, and identifying the protein product encoded thereby, thereby identifying a protein which is associated with depression or other behavioral disorders in the mammal.

The preferred tissue from which the nucleic acid is obtained for use in the above-described screening assay is selected from the group consisting of whole brain, amygdala, prefrontal cortex, hippocampus and other limbic brain regions. The most preferred tissue is the amygdala, prefrontal cortex, or hippocampus.

Reverse transcriptase-polymerase chain reaction (RT-PCR)/differential display may also be used to identify nucleic acids and proteins associated with depression, as described herein in the experimental examples.

The invention thus also includes isolated nucleic acids, isolated genes, isolated proteins and isolated polypeptides which are identified using the methods described herein.

The invention includes isolated nucleic acids having the sequences disclosed in SEQ ID NO: 1 and SEQ ID NO: 3, respectively. SEQ ID NO: 1 represents a rat nucleotide sequence encoding the mss4 protein and SEQ ID NO: 3 represents a nucleotide sequence encoding the rat mitochondrial ATP synthase subunit 6 protein. The human counterparts are listed in SEQ ID NO: 2 and SEQ ID NO: 4.

Once a nucleic acid associated with depression or other behavioral disorders, and the protein encoded thereby is identified, it is well within the skill of the artisan to develop compounds which have therapeutic value with respect to treatment of depression and behavioral disorders in mammals. For example, antisense compounds may be generated using a knowledge of the nucleic acid and its expression in cells, which antisense compounds are useful for inhibiting expression of the nucleic acid in the cell of a mammal in need of treatment for depression and/or behavioral disorders. Thus, antisense compounds are useful when increased expression of a nucleic acid is associated with depression or other behavioral disorders, or when the expression per se of a particular nucleic acid is associated with depression or other behavioral disorders.

The generation of effective antisense compounds is well known in the art and is described for example, in U.S. Pat. No. 5,034,506 and in Nielsen et al. (1991, Science 254:1497).

It is an aspect of this invention to include antisense compounds to SEQ ID NOs: 1-4.

Other compounds which have therapeutic value are those that modulate the activity of polypeptides of the invention. It is well within the skilled artisan's knowledge to develop and/or test compounds which modulate the level of expression of a protein or the activity of the expressed polypeptide of the invention. For example, inhibitors or enhancers may be developed using the polypeptides identified by the methods of the present invention, which inhibitors or enhancers are therapeutic when administered to a mammal having increased or decreased expression of any of the polypeptides identified by the methods of the present invention.

In addition, depending on the function of the nucleic acid identified in the just-described screening assays, a nucleic acid involved in depression and behavioral disorders may be beneficial to a mammal when administered to the mammal in a sense orientation. Sense nucleic acids are useful when decreased expression of a nucleic acid is associated with depression or other behavioral disorders, or when the absence of expression per se of a particular nucleic acid is associated with depression or other behavioral disorders.

Antisense and sense nucleic acids may be delivered to the mammal as “naked” nucleic acid, or may be delivered to the mammal in the form of a vector, either in a viral or a non-viral vector as defined herein.

Nucleic acids associated with depression or other behavioral disorders, as described herein, are also useful for diagnosis of depression or other behavioral disorders. It is a simple matter to obtain tissue from a mammal suspected of having depression or other behavioral disorders and to identify expression of nucleic acid in the cells therein using nucleic acid obtained as described herein as a probe.

Nucleic acids associated with depression or other behavioral disorders, as described herein, are also useful as targets for drug discovery. If gene expression is upregulated in depressed rats, it is a simple matter to screen for compounds which will downregulate expression of that particular gene. Likewise, if a gene is downregulated in depressed rats, compounds can be screened for upregulation of expression of that gene. Thus, new drugs can be developed which can treat and/or alleviate depression or other behavioral disorders by regulating expression of genes associated with such disorders.

The proteins and peptides encoded by nucleic acids involved in depression or other behavioral disorders in a mammal are also useful for diagnosis and treatment of depression or other behavioral disorders in a mammal. Such proteins and polypeptides may be used to generate a variety of antibodies as defined herein, which antibodies may be used to either diagnose or treat depression and behavioral disorders. Diagnosis of a disease using an antibody is common in the art as is treatment of disease using antibodies.

Proteins and peptides which are associated with behavioral disorders may also be useful for the generation of peptidomimetics and other small molecules useful for treatment of such disorders. Peptidomimetics may be generated using techniques described in PCT/US93/01201 and in U.S. Pat. No. 5,334,702.

Proteins encoded by SEQ ID NOs: 1-4 are examples of proteins associated with depression and/or behavioral disorders.

Cells derived from the rats of the invention are also useful for the identification of compounds useful for treatment of depression and behavioral disorders. Suitable cells include, but are not limited to, primary neuronal cultures, primary anterior pituitary cultures, and lymphocyte and monocyte cultures of cells. Such cells may be obtained from tissues of both WMI and WLI rats and may be cultured in vitro, as primary cells, or even as more stable cell lines. These cells are chosen because they differentially express, or do not express, as the case may be, nucleic acids which are associated with depression or other behavioral disorders. The effect of a test compound on the inhibition or activation of expression of a nucleic acid, or on the level of expression of the nucleic acid, or on the function of a protein product encoded by that nucleic acid, is then assessed by measuring expression of the nucleic acid in the cells, or of assessing the function of the protein. The in vitro screening assay just described need not necessarily be confined to the use of a cell, but may also include the use of a tissue obtained from the WMI or the WLI rat of the invention as disclosed herein.

Thus, the invention includes a cell obtained from either a WMI or a WLI rat and a tissue obtained from a WMI or a WLI rat for the purpose of identifying compounds which affect behavioral disorders in mammals.

Compounds which are identified using any of the methods described herein may be formulated and administered to a mammal for treatment of a behavioral disorder as now described.

The invention encompasses the preparation and use of pharmaceutical compositions comprising a compound useful for treatment of a behavioral disorder as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats and dogs

Pharmaceutical compositions that are useful in the methods of the invention may be prepared, packaged, or sold in formulations suitable for oral, parenteral, pulmonary, intranasal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0. 1% (w/w) and as much as 100% (w/w) of the active ingredient, and may further comprise one or more of the additional ingredients described herein.

Typically dosages of the compound of the invention which may be administered to an animal, preferably a human, range in amount from 1 g to about 100 g per killogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per killogram of body weight of the animal. More preferably, the dosage will vary from about 10 mg to about 1 g per killogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Definitions

The following terms have the following meaning as used herein in the present application.

The articles “a” and “an” are used herein to refer to one or to more than one (i.e. to at least one) of the grammatical object of the article. By way of example, “an element” means one element or more than one element.

The term “antibody,” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (Harlow et al., 1988, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “synthetic antibody” refers to an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “behavioral disorder” in a mammal should be construed to include, but not be necessarily limited to, the following behaviors: major depression, bipolar depression, generalized anxiety disorder, obsessive compulsive disorder, post-traumatic stress disorder, dysthymia, cyclothymia and premenstrual syndrome.

The term “depression” means major depression as defined by the DSM-IV criteria (American Psychiatric Association: Diagnostic and Statistical Manual of Mental Disorders, Fourth Edition, Washington, D.C. American Psychiatric Association, 1994).

Behaviors which are measured in an animal as an indication of a behavioral disorder include, but are not limited to, behaviors measured in the following tests: the FST, the light/dark box test, the elevated plus maze test, the locomotor activity test, and the open field behavior test. Essentially, those of skill in the art would know that other routine behaviors may be measured using ordinary protocols available in the art.

The term “differential gene expression” means the expression of a gene in one cell type compared with the absence of expression of the gene in a similar cell type, wherein one cell is obtained from one animal or tissue, and the other cell is obtained from a different tissue in the same animal, or from a different animal, the animals or tissues being referred to herein as a “system.” The term also means the absence of expression of a gene in one system compared with the expression of the gene in another system. In addition, the term should be construed to mean a difference in the level expression of a gene in one system versus another system.

“Alleviating depression or a behavioral disorder” means reducing the severity of the depression or behavioral disorder.

“Treating depression or a behavioral disorder” means reducing the frequency or amplitude with which a symptom of the depression or behavioral disorder is experienced by a patient.

The term “antisense oligonucleotide” means a nucleic acid polymer, at least a portion of which is complementary to a nucleic acid which is present in a normal cell or in an affected cell. The antisense oligonucleotides of the invention preferably comprise between about fourteen and about fifty nucleotides. More preferably, the antisense oligonucleotides comprise between about twelve and about thirty nucleotides. Most preferably, the antisense oligonucleotides comprise between about sixteen and about twenty-one nucleotides. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497).

“Antisense” also refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “founder rat” as used herein means the F1 progeny of commercially available WKY rats.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

An “mRNA-coding region” of a gene consists of the nucleotide residues of the coding strand of the gene and the nucleotide residues of the non-coding strand of the gene which are homologous with or complementary to, respectively, an mRNA molecule which is produced by transcription of the gene. It is understood that, owing to mRNA processing which occurs in certain instances in eukaryotic cells, the mRNA-coding region of a gene may comprise a single region or a plurality of regions separated from one another in the gene as it occurs in the genome. Where the mRNA-coding region of a gene comprises separate regions in a genome, “mRNA-coding region” refers both individually and collectively to each of these regions.

A “coding region” of an mRNA molecule consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g. amino acid residues in a protein export signal sequence).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, an “essentially pure” preparation of a particular protein is a preparation wherein at least about 95%, and preferably at least about 99%, by weight, of the protein in the preparation is the particular protein.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The invention is now described with reference to the following experimental Examples. These Examples are provided for the purpose of illustration only and the invention should in no way be construed as being limited to this Example, but rather should be construed to encompass any and all variations which become evident as a result of the teaching provided herein.

### EXAMPLE 1

### Generation and Characterization of WMI and WLI Rats

In the experiments performed in the present Example, Wistar Kyoto (WKY) rats, which are a commercially available inbred strain, were segregated into two groups, more depressed and less depressed rats. The more depressed rats were bred with each other, and the less depressed rats were also bred with each other. At about 60 days of age, i.e., when the rats were young adults, the rats were tested for depressive behavior as described in detail herein, and those progeny rats exhibiting more depressive behavior were selected for further breeding to generate a colony of WKY-More Immobile (WMI) rats. Similarly, those progeny rats exhibiting less depressive behavior were also selected for further breeding to generate a colony of WKY-Less Immobile (WLI) rats. At least F1 generation, and preferably F2 generation colonies of WMI and WLI rats were thus obtained which are the subject of the present invention.

Essentially, the FST test was used to establish a phenotype within commercially available WKY rats. Based on FST immobility scores, male and female rats were selected having high (more immobile) and low (less immobile) FST immobility scores. One male rat, named 6H2 and indicated by the * in the Table presented herein, exhibited a high FST immobility score of 13. Rat 6H2 was mated with a female rat named F4, exhibiting an FST immobility score of 21 (also indicated by the * in the Table). The male progeny of this mating had average FST immobility scores of 24.9+/−4.5 and the female progeny of this mating had average FST immobility scores of 27.3+/−3.4. Thus, all F1 generation or higher rats obtained from this mating were even more depressed than either of the parent rats. These F1 generation or higher rats are referred to herein as WMI rats and are the subject of the present invention.

Similarly, one male rat, named 3H1 and indicated by the ^{@} in the Table presented herein, exhibited a low FST immobility score of 7. Rat 3H1 was mated with a female rat named F6, exhibiting an FST immobility score of 6 (also indicated by the ^{@} in the Table). The male progeny of this mating had average FST immobility scores of 3.0+/−0.8 and the female progeny of this mating had average FST immobility scores of 2.8+/−1.1. Thus, all F1 generation or higher rats obtained from this mating were even less depressed than either of the parent rats. These F1 generation or higher rats are referred to herein as WLI rats and are also the subject of the present invention.

The characteristics of the rats of the invention are presented in the following Table.

| Parents/Immobility scores | MALES | FEMALES |
|---|---|---|
| Male: 11; Female: 19 | 12.6 +/− 2.7 (n = 8) | 14.75 +/− 3.6 (n = 4) |
| Male: 13; Female: 21* | 24.9 +/− 4.5 (n = 8) | 27.3 +/− 3.4 (n = 3) |
| More Immobile | More Immobile | More Immobile |
| Male: 17; Female: 32 | 14.3 +/− 5.8 (n = 6) | 16.8 +/− 2.2 (n = 4) |
| Male 15; Female: 27 | 4.8 +/− 2.8 (n = 4) | 18.7 +/− 4.3 (n = 7) |
| Male: 17; Female: 27 | 10.4 +/− 3.4 (n = 5) | 19.2 +/− 3.2 (n = 9) |
| Male: 7; Female: 6^{@} | 3.0 +/− 0.8 (n = 7) | 2.8 +/− 1.1 (n = 5) |
| Less Immobile | Less Immobile | Less Immobile |

*The progeny of this mating forms the WMI rat colony.
^{@}The progeny of this mating forms the WLI rat colony.

Unselected parent WKY rats also exhibit a dramatically and significantly prolonged circadian peak of plasma ACTH and corticosterone levels compared with Wistar rats. Thus, the WKY rats exhibits behavioral and physiological (i.e., the disruption of the circadian patterns of plasma glucocorticoids) similarities to humans with respect to depressive disorders and thus are a useful animal model for the study of depressive disorders in mammals.

### EXAMPLE 2

### Detailed Characterization of WMI and WLI Rats

WMI and WLI rats were further characterized as follows.

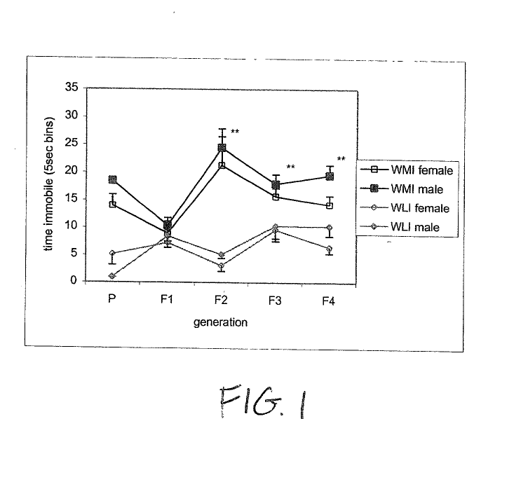

In the first set of experiments, the immobility scores for the parent WKY rats and the selectively bred substrain WMI and WLI rats were assessed. The animals were tested as adults at 60 days of age. Males and females having similar high (more immobile) and low (less immobile) immobility scores were selected and were mated with each other (i.e., more immobile rats were mated together and less immobile rats were mated together). The resulting F1 generation animals were tested in the FST and three brother/sister breeding pairs from the litter that consistently displayed the highest immobility in the FST for WMI rats, and three brother/sister breeding pairs from the litter that consistently displayed the lowest immobility in the FST for WLI rats were selected for further breeding. The resulting F2 generation animals in each category (WMI and WLI) were tested in the FST and the results are shown in FIG. 1. As can be seen in the Figure, the rats bred true with respect to the FST scores.

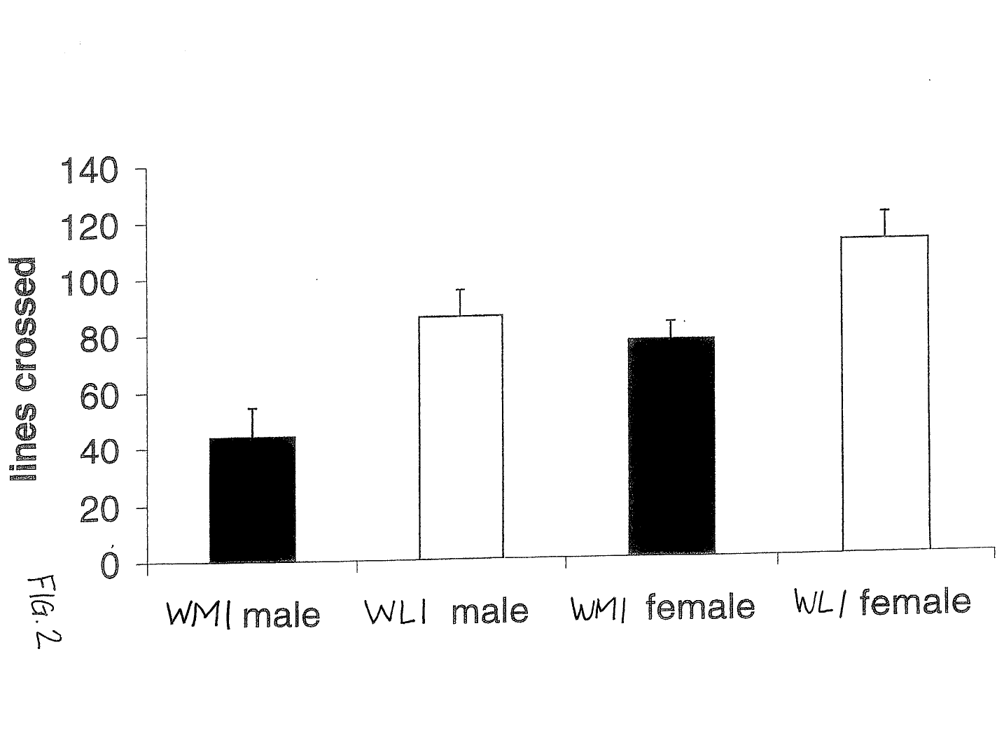

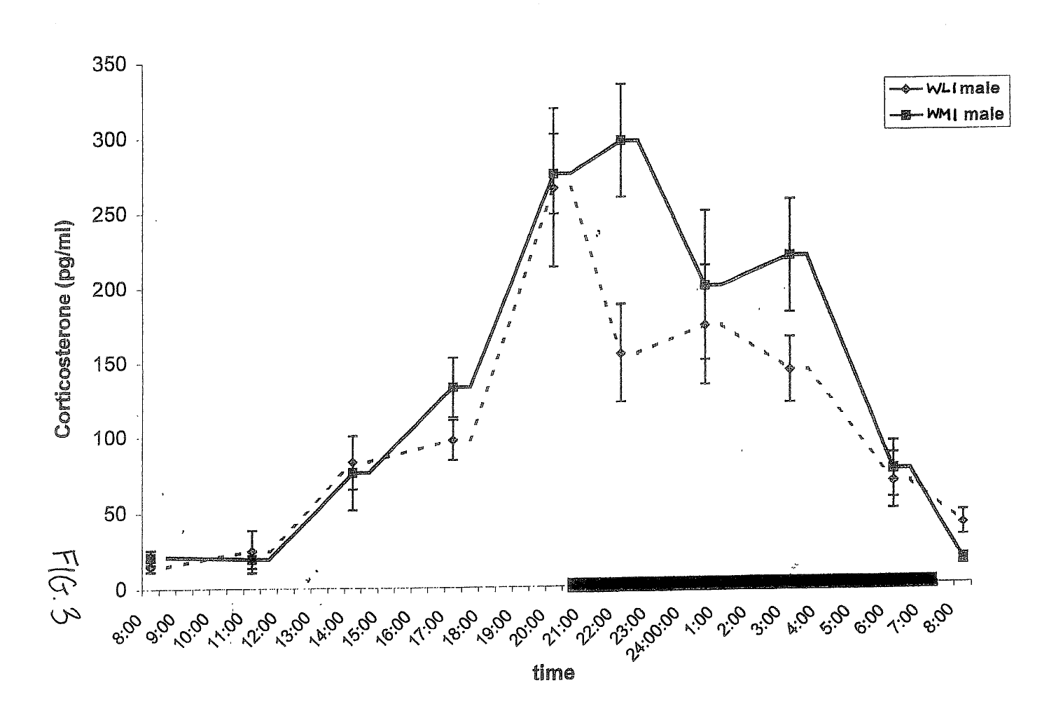

In a similar experiment, the F1 generation rats were tested as adults at 60 days of age in the open field test. This test took place on these animals at least one week after the FST was performed. As the data shown in FIG. 2 illustrate, the WMI animals exhibited significantly less ambulation than did the WLI animals.

A plasma corticosterone profile of FI generation WMI and WLI rats was obtained. In this experiment, three month old WMI and WLI male rats were implanted with jugular cannulae to allow for 24 hour serial blood sampling. The surgery took place two weeks after the animals were placed on a 14 hour:10 hour light:dark cycle. Blood samples (0.7 ml) were taken every 2-3 hours over a 24 hour period two days after the surgery. Radioimmunoassay was used to measure hormone levels. Plasma corticosterone levels at 2200 hours were significantly higher in the F1 generation WMI male animals compared with the WLI male animals (FIG. 3).

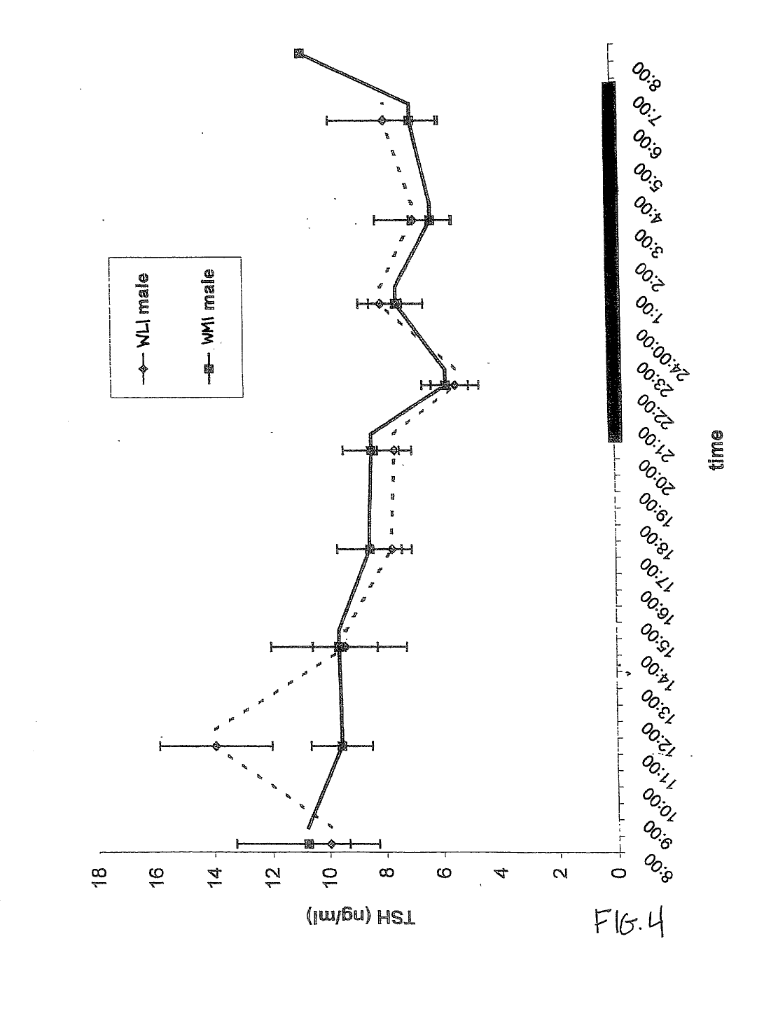

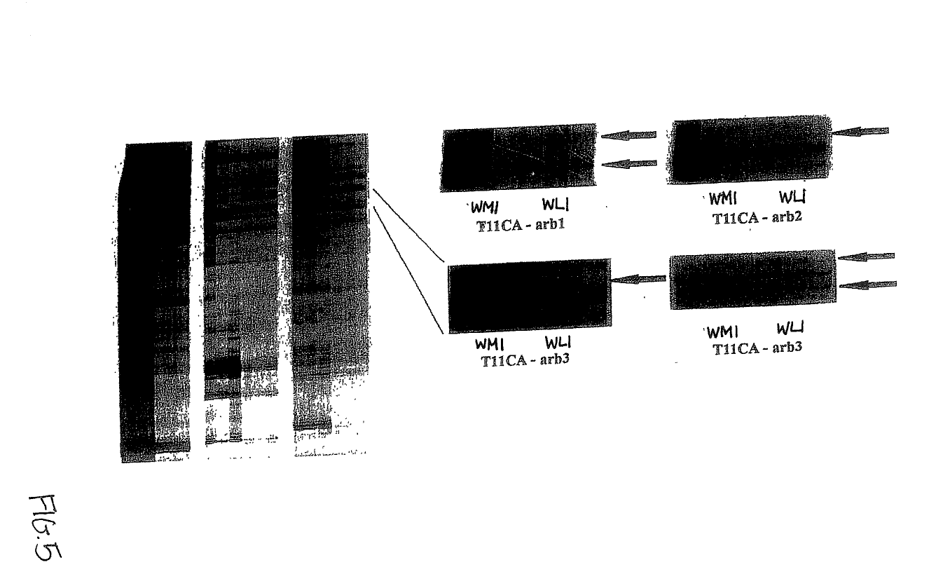

In a similar experiment, the plasma TSH profile was assessed in the animals as described herein with respect to FIG. 3. These data are shown in FIG. 4. As can be seen in FIG. 4, the TSH levels in the WLI animals peaked at about 1100 hours as is characteristic of animals having a normal circadian profile of plasma TSH. In contrast, the WMI animals exhibited an absence of this diurnal peak, which is similar to the lack of a diurnal TSH peak observed in depressed human patients.

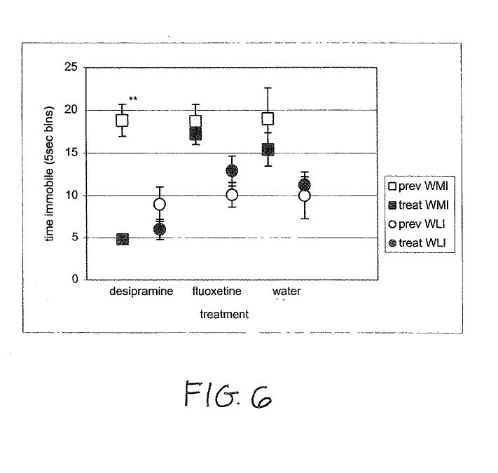

To examine mRNA expression in these animals, differential display RT-PCR analysis was performed on mRNA obtained from amygdala of the parental generation of breeding mothers of the WMI and WLI animals. Differential display was performed as described in Berger et al. (1997, Soc. for Neurosci. Vol. 23(1):853). Essentially, RNA was extracted from the tissue and samples were amplified using one anchor primer (T11CA, Genhunter Corp., Nashville, Tenn.) in combination with three arbitrary primers and the products were electrophoresed. Ten bands were identified as being differentially expressed (FIG. 5) in the amygdala of WMI rats compared with WLI rats.

Thus, this type of experimentation performed on selectively bred rats can lead to the identification of genes which are involved in depression and other behavioral disorders.

### EXAMPLE 3

### Isolation of Nucleic Acids from WMI and WLI Rats

Nucleic acids related to behavioral disorder were isolated from WMI and WLI using subtractive suppressive polymerase chain reaction (PCR). The materials and methods follow.

Adult WKY males were selected from the parental generation and bred WMI and WLI. Rats were first scored using the FST. Rats categorized as WMI had a mean FST immobility score of 20.8+/−3.6 and rats categorized as WLI had a mean FST immobility score of 3.8+/−0.6.

Rats were decapitated under basal conditions, i.e., unstressed, within two minutes of initial contact and amygdala was dissected according to Paxinos coordinates (1997, Paxinos and Watson, The Rat Brain in Stereotaxtic Coordinates, 3rd Ed., Academic Press, San Diego, Calif.). A brain block (Harvard Apparatus, Holliston Mass.) was used for the amygdala dissection, and cuts were −0.80 to −4.16 from the bregma.

Total RNA was isolated, treated with DNase, and concentration was measured using spectrophotometry. Amygdala mRNA obtained from five WMI males was pooled, evenly divided into two pools, and mRNA obtained from five WLI males was pooled and evenly divided into two pools. Each mRNA pool was reverse transcribed into cDNA using the CLONTECH SMART PCR cDNA™ Synthesis Kit following the manufacturer's instructions. The CLONTECH PCR-SELECT™ cDNA Subtraction kit was then used to produce clones from the four cDNA pools. Each cDNA pool was used both as tester and driver DNA. A phenol/chloroform/isopropanol extraction with salt/ethanol precipitation was used instead of the manufacturer's recommended chromacolumn purification, and 3 microliters of template instead of the recommended one microliter of template was used.

The subtractive cloning produced 126 clones selected by higher levels of expression in WLI than in WMI and 261 clones selected by higher levels of expression in WMI than in WLI. These 387 clones were screened by pairwise comparison of expression using amygdala RNA from individual WMI and WLI males. The clones were grown on filters. Each filter was probed with radiolabeled reverse strand primed RNA of individual male WMI and WLI amygdala. Two of the five WMI males were selected from the parental generation, two from the F1 generation, and one from the F2 generation. These animals had a mean FST of 25+/−5.8. One of the five WLI males was selected from the parental generation, one from the F1 generation, two from the F2 generation and one from the F3 generation. These animals had a mean FST of 2.6+/−0.8. All 387 clones were analyzed against RNA obtained from these ten animals. Results were exposed onto an autoradiographic film and scanned into Adobe Photoshop 5.5. Optical density measurements were taken with NIH Image 1.6. Optical density readings were normalized by volume and WMI/WLI ratios were taken. Clones that demonstrated greater expression in the expected direction in every comparison were selected for sequencing.

Clones were sequenced and aligned. Homology searches were performed using the NCBI GenBank website (http://www.ncbi.nlm.nih.gov/BLAST) and primer pairs were chosen using Web Primer, located on the Saccharomyces Genome Database website (http://www.genome-www.stanford.edu/Saccharomyces).

Five confirmation rounds of PCR were conducted, indicating two genes having consistently higher expression in the amygdala of the WMI animals compared to the WLI animals: the mitochondrial ATP synthase subunit 6 (SEQ ID NOs: 1 and 2)and a mammalian guanine nucleotide releasing protein, mss4 (SEQ ID NOs: 3 and 4). These genes were upregulated in the parental, F1, and F2 generations of selectively bred WMI substrains.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A WMI rat, wherein said rat exhibits a forced swim test immobility score of greater than about 9.

2. The WMI rat of claim 1, wherein said rat exhibits a forced swim test score of greater than about 11.

3. A WLI rat, wherein said rat exhibits a forced swim test immobility score of lower than about 8.

4. The WLI rat of claim 3, wherein said rat exhibits a forced swim test immobility score of lower than about 6.

5. A method of identifying a compound capable of treating a behavioral disorder in a mammal, said method comprising administering a test compound to a WMI rat, administering a placebo to an otherwise identical WMI rat, and assessing a behavior of each of said rats, wherein a change in said behavior of said rat administered said test compound, compared with the behavior of said rat administered said placebo, is an indication that said test compound is capable of treating a behavioral disorder in a mammal.

6. The method of claim 5, wherein said mammal is resistant to tricyclic and serotonergic antidepressants.

7. The method of claim 5, wherein said compound is a fast-acting compound.

8. The method of claim 5, wherein said behavioral disorder is alleviated.

9. The method of claim 5, wherein said behavior is assessed in a test selected from the group consisting of a forced swim test, an elevated plus maze test, a light/dark box test, an open field behavior test and a locomotor behavior test.

10. The method of claim 5, wherein said mammal is a human.

11. The method of claim 1O, wherein said behavioral disorder is selected from the group consisting of major depression, bipolar depression, generalized anxiety disorder, obsessive compulsive disorder, post-traumatic stress disorder, dysthymia, cyclothymia and premenstrual syndrome.

12. A method of identifying a compound capable of treating a behavioral disorder in a mammal, said method comprising administering a test compound to a WLI rat, administering a placebo to an otherwise identical WLI rat, and assessing a behavior of each of said rats, wherein a change in said behavior of said rat administered said test compound, compared with the behavior of said rat administered said placebo, is an indication that said test compound is capable of treating a behavioral disorder in a mammal.

13. The method of claim 12, wherein said compound is a fast-acting compound.

14. The method of claim 12, wherein said mammal is resistant to tricyclic and serotonergic antidepressants.

15. The method of claim 12, wherein said behavioral disorder is alleviated.

16. The method of claim 12, wherein said behavior is assessed in a test selected from the group consisting of a forced swim test, an elevated plus maze test, a light/dark box test, an open field behavior test and a locomotor behavior test.

17. The method of claim 12, wherein said mammal is a human.

18. The method of claim 17, wherein said behavioral disorder is selected from the group consisting of major depression, bipolar depression, generalized anxiety disorder, obsessive compulsive disorder, post-traumatic stress disorder, dysthymia, cyclothymia and premenstrual syndrome.

19. A method of isolating a nucleic acid associated with a behavioral disorder in a mammal, said method comprising comparing mRNA obtained from a tissue in a WMI rat with mRNA obtained from the same tissue in a WLI rat and isolating nucleic acid corresponding to mRNA expressed in said WMI rat, which mRNA is either not expressed in said WLI rat or is expressed at a substantially lower level in said WLI rat, thereby isolating a nucleic acid associated with a behavioral disorder in said mammal.

20. The method of claim 19, wherein said mammal is resistant to tricyclic and serotonergic antidepressants.

21. The method of claim 19, wherein said comparison of said mRNAs is conducted by differential screening of libraries which are generated using said mRNAs.

22. The method of claim 21, wherein said comparison is conducted by suppressive, subtractive PCR.

23. The method of claim 19, wherein said comparison is conducted using reverse transcriptase PCR differential display.

24. The method of claim 19, wherein said tissue is selected from the group consisting of whole brain, amygdala, prefrontal cortex, hippocampus, and limbic brain region.

25. The method of claim 19, wherein said tissue is selected from the group consisting of amygdala, prefrontal cortex, and hippocampus.

26. A method of isolating a nucleic acid associated with a behavioral disorder in a mammal, said method comprising comparing mRNA obtained from a tissue in a WMI rat with mRNA obtained from the same tissue in a WLI rat and isolating nucleic acid corresponding to mRNA which is either not expressed in said WMI rat or which is expressed in said WMI rat at a substantially lower level than expression of said nucleic acid in said WLI rat, thereby isolating a nucleic acid associated with a behavioral disorder in said mammal.

27. The method of claim 26, wherein said mammal is resistant to tricyclic and serotonergic antidepressants.

28. The method of claim 26, wherein said comparison of said mRNAs is conducted by differential screening of libraries which are generated using said mRNAs.

29. The method of claim 27, wherein said comparison is conducted by suppressive, subtractive PCR.

30. The method of claim 26, wherein said comparison is conducted using reverse transcriptase PCR differential display.

31. The method of claim 26, wherein said tissue is selected from the group consisting of whole brain, amygdala, prefrontal cortex, hippocampus, and limbic brain region.

32. The method of claim 31, wherein said tissue is selected from the group consisting of amygdala, prefrontal cortex, and hippocampus.

33. A nucleic acid associated with a behavioral disorder in a mammal isolated by the method of claim 19.

34. An antisense nucleic acid which corresponds to the nucleic acid of claim 33.

35. A nucleic acid associated with a behavioral disorder in a mammal isolated by the method of claim 26.

36. An antisense nucleic acid which corresponds to the nucleic acid of claim 35.

37. A method of isolating a polypeptide associated with a behavioral disorder in a mammal, said method comprising comparing mRNA obtained from a tissue in a WMI rat with mRNA obtained from the same tissue in a WLI rat and isolating nucleic acid corresponding to mRNA expressed in said WMI rat, which mRNA is either not expressed in said WLI rat or is expressed at a substantially lower level in said WLI rat, thereby isolating a nucleic acid associated with a behavioral disorder in said mammal and obtaining the polypeptide encoded thereby.

38. The method of claim 37, wherein said mammal is resistant to tricyclic and serotonergic antidepressants.

39. The method of claim 37, wherein said comparison of said mRNAs is conducted by differential screening of libraries which are generated using said mRNAs.

40. The method of claim 39, wherein said comparison is conducted by suppressive, subtractive PCR.

41. The method of claim 37, wherein said comparison is conducted using reverse transcriptase PCR differential display.

42. The method of claim 37, wherein said tissue is selected from the group consisting of whole brain, amygdala, prefrontalcortex, hippocampus, and limbic brain region.

43. A polypeptide associated with a behavioral disorder in a mammal isolated by the method of claim 37.

44. An antibody which specifically binds to the polypeptide of claim 43.

45. A compound that modulates the activity of the polypeptide of claim 43.

46. A method of isolating a polypeptide associated with a behavioral disorder in a mammal, said method comprising comparing mRNA obtained from a tissue in a WMI rat with mRNA obtained from the same tissue in a WLI rat and isolating nucleic acid corresponding to mRNA which is either not expressed in said WMI rat or which is expressed in said WMI rat at a substantially lower level than expression of said nucleic acid in said WLI rat, thereby isolating a nucleic acid associated with a behavioral disorder in said mammal and obtaining the polypeptide encoded thereby.

47. The method of claim 46, wherein said mammal is resistant to tricyclic and serotonergic antidepressants.

48. The method of claim 46, wherein said comparison of said mRNAs is conducted by differential screening of libraries which are generated using said mRNAs.

49. The method of claim 48, wherein said comparison is conducted by suppressive, subtractive PCR.

50. The method of claim 46, wherein said comparison is conducted using reverse transcriptase PCR differential display.

51. The method of claim 46, wherein said tissue is selected from the group consisting of whole brain, amygdala, prefrontal cortex, hippocampus, and limbic brain region.

52. A polypeptide associated with a behavioral disorder in a mammal isolated by the method of claim 37.

53. An antibody which specifically binds to the polypeptide of claim 52.

54. A compound that modulates the activity of the polypeptide of claim 52.

55. An isolated cell obtained from a WMI rat.

56. An isolated cell obtained from a WLI rat.

57. A method of identifying a compound that regulates expression of a nucleotide sequence associated with depression or a behavioral disorder in a mammal, said method comprising contacting a cell from said mammal with said compound and determining whether the level of expression of said nucleotide sequence is altered in the presence of said compound compared with the level of expression of said nucleotide sequence in the absence of said compound, wherein a higher or lower level of expression in the presence of said compound in an indication that said compound regulates expression of said nucleotide sequence.

58. The method of claim 57, wherein said nucleotide sequence is selected from the group consisting of SEQ ID NOs: 1-4.

59. A method of identifying a nucleic acid associated with depression or a behavioral disorder in a mammal, said method comprising isolating mRNA from a tissue in a WMI rat and isolating mRNA from the same tissue in a WLI rat, reverse transcribing said mRNAs to their respective cDNAs, performing subtractive suppressive PCR using the cDNA as both testers and drivers, and identifying those mRNAs exhibiting increased or decreased expression in tissue from WMI rats compared with tissue from WLI rats, thereby identifying said nucleic acid.

60. The method of claim 59, wherein said tissue is selected from the group consisting of whole brain, amygdala, prefrontal cortex, hippocampus, and limbic brain region.

61. The method of claim 60, wherein said tissue is selected from the group consisting of amygdala, prefrontal cortex, and hippocampus.

62. A nucleic acid associated with a behavioral disorder in a mammal isolated by the method of claim 59.

63. An antisense nucleic acid which corresponds to the nucleic acid of claim 62.


SEQUENCE LISTING (4 sequences)

SEQ ID NO: 1 - DNA, 2500 bp, Rattus norvegicus
ggtagccactttggacttatcttcaagaatgtgacatttgtggtaatattctttgcttccccatggcctcaccgtgccacaacagtgttacgtgtttgacactcactgcttgtgcacccatcacttctgatgtatggcagttttgggtcaataagatattagtattactttgactttgacttgtgagtctaggttaaaatcattcggaggattttttattctccgaggtcaccccaaccgaaattttttagttcatatttattttgttttagcccattaggttgtttttatataattgaactagtaaattaaagctccatagggcttctccgtcttattgggagattccagcctcttcactggaaggtcaatttcactgattgaaagtaagagacagttgaaccctcgtttagccattcattctagtccctaattaaggaacaagtgattatgctacctttgcacggtcaggataccgcggccgtttaactttagtcactgggcaggcaatgctctaatacttgttatgctagaggtatgtttttggtaaacaggcggggttcatgtttgccgagttccttttactttttaatctttccttaaagcacgcctgtgttgggctaacgagttagggataggtaattctattgttgggttagtacctatgattcgataattgacaatggttatccgggttgtcatacacttgtgctaggagaggcaatggaaccctgcgagctgcagaacgagctagtgtcagccgaaggccggaaccggaaggcggtgctgtgccagcgttgtggctctcgggtgctgcagccagggactgctcttttctctcgccggcagcttttcctcccctccatgaggaagaagccggatctggtggacggcagcaatcctgatggtgatgtcctcgaggaacactggctggttaatgacatgttcatctttgagaacgtgggctttaccaaggacgtgggcaacgtcaagtttctagtctgtgcagactgtgagattggacccattggctggcattgcctagatgacaagaacagcttctatgtggccttggagcgagtttcccacgagtaactgaggggtttgggacttggctccaccctagctataaagctctgcccacaagaaccgacactgatgtgtcagcagtgctggcagacttcttggtactaatataagcaccggtgtttgcccttgaacttgcagagggccggtcctgtttcctcaggcctcagagcatgtctcctacttagtcctctctgtcacatgtgtcctagagccctttcccctgacttggaacatggaagaacacgcgagtgctctaccagtgtagtctggttccggcactctgcctccgcacctcgatgccaagctctcttcttctcagcaagaacaacattcatctttagccatcacgacgggaactaagatgctaatatttgaaactccacctttcctcattctagaagccaagtgtgatagggtgcacatctgtaatcctaggtcttgagtgggtagaggcaggaagatcaggagttcaagggcagccttagctacatagactgggccaagtgagatgttgtctcaaaaaacataaagcatacaagtatttgagagtaatctcattgtatgtgacctacttatctacagtaatttattctttttaaaagtaaaacatttacagatgtcagtatttaaacctcactttagatacatttttagtactgtgtttgagagccttttttccgggtcaatagtggcattttataatgaggacgaacacgactcctcttccctcagatcttgatcaggtttctctttttagtcattccttgcccctgatgtcaaccctaaggaaaactcagccctagctgtgtgccagactctcttgtaaacctgttccacggacatttctattgttatggacaaggtgttaccttttcttattattagccaacttaaatcttcaatatgatttccagttactctgtcatttctcttctggagtgactcagtagcttaaaaggactctgtgtaaacaggttcaaatactgtgtcatcatcccctcctctgagggcctaaattgagataagtagagacttcattattcctcatgccagagcttaggaagtgggggcagatcaagagttcaaggtcagcctcagttactataatatcaaattcaaggccagcctgtactatggagatcttgtttcaaatgaacaaaacacaaaataaacaagtaggacctgcaaaagtgacacgtagttaaaaatgtttgctgctctttcaggcgacctgagttcaggaccttgcaccctcatgggaggtttattccagttcaaggagatctgcgccttctggtccctgcagatgccttcatgtgtgtggcacccatacaaacaagtgagcacactcgcgtacacacacacacacgcacgcacgcacgtgaataaaaattttacaaaaaaaaaaaaa

SEQ ID NO: 2 - DNA, 922 bp, Homo sapiens
ccggctgcggcgatggaaccagcggacgagccgagcgagttagtgtcagccgagggccgaaaccggaaggcggtgctgtgccagcgttgcggctcccgggtgctgcagccagggaccgctctcttctctcgccgacagcttttccttccctccatgagaaagaagccagctctgtctgacggcagcaatcctgacggcgatctcctccaggaacactggctggttgaggacatgttcatttttgagaatgtgggcttcaccaaggacgtgggcaacatcaagtttctggtctgcgcagactgtgaaattggaccaattggctggcattgcctagatgacaagaacagtttctatgtggccttggaacgagtttcccatgagtaactgaggggaggggtactcagctccatctccaaagataaacctactccccacaagaactggcctttaatgtggtataactgttccgctgccttcttgtctgtgtgctaatataaatactgagtaccagcatgtccatttgaacatgcaaagggttaatcctgcttcctaaagcctcaagtacatgcctcctgcttagttcactttgtatcacatttcctaagctcccttttcccccagttttgggacactgtgcttacctccaaaaatctcatctcttccctggcattctccctaggctctgttttgcccagggctcccgctttttcttgctctagaggagcagtattcaaccttttagctatgatgacacataacaaaagatgtttatgtactaatagttgaaatctgcctttttctcattcaagaaggcatacaaatatctgagagtgactttgttgtatggctacccttgtgatctacagtaatttattctttctaaaagtaaagcattctcaaaacaaaaaaaaaaaaaaaaagg

SEQ ID NO: 3 - DNA, 961 bp, Rattus norvegicus
gcgaagcttagagcgttaaccttttaagttaaagttagagacaacaaatctccacaatgacatgccacaactagacacatccacatgatttattacaatcatctcctcaatagccacactatttattttatttcaattaaaaatttcttcccaaacctttcctgcacctccctcacccaaaactatagccacagaaaaaacgaataacccttgagaatcaaaatgaacgaaaatctatttgcctctttcattacccccacaataataggtctaccaattgttgtgaccattattatgttcccatcaattctattcccatcatcagaacgcctaatcagcaaccgactacactcatttcaacactgactaatcaaacttatcatcaaacaaataatgttaatccacacaccaaaaggacgaacctgagccctaataattgtatccctaattatatttattggctcaaccaaccttctagggcttcttccccatacatttacccctaccactcagctatctataaacctaagcatagccatccccctatgagcaggagccgtaattctaggcttccgacacaaactaaaaaattctttagcccacttctcaccgcaaggaacccccatctcactaattcccatactaatcatcatcgaaactatcagcctatttattcaaccgatagcactagcagtacgactaacagcaaacattacagcaggccatctattaatgcatctaatcggaggagctaccctagtacttatagacatcagcccaccaaccgctacaattacatttattattctacttctacttacagtacttgaatttgccgtagccttaattcaagcctatgtattcacccttctagtaagcctgtacctacatgataacacataatgacccaccaaacccatgcataccatatagtaaacccaagcccatgaccactaacagg

SEQ ID NO: 4 - DNA, 681 bp, Homo Sapiens
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataa